(19) United States
(12) Patent Application Publication
(10) Pub. No.: US 2020/0179457 A1
(43) Pub. Date: Jun. 11, 2020

(54) Zeus Protocol Method for Enhancing Male Sexual Wellness with Shockwave Therapy and Stem Cells

(71) Applicant: Joy Kong, Chatsworth, CA (US)

(72) Inventor: Joy Kong, Chatsworth, CA (US)

(21) Appl. No.: 16/215,982

(22) Filed: Dec. 11, 2018

(51) Int. Cl. A61K 35/28; A61P 15/10; A61P 15/08; A61B 17/22; A61B 17/225; A61H 23/00

(52) U.S. Cl. CPC A61K 35/28; A61P 15/10; A61H 23/008; A61B 17/22004; A61B 17/225; A61P 15/08

(57) ABSTRACT

A method for enhancing male sexual wellness with shockwave therapy and stem cells promotes tissue repair and regeneration in or near the male penis, through the combinative effects of shockwave therapy and needle-free injection of a stem cell specimen on the penile surface of the penis. The method includes: preparing the male patient, both mentally and physically to receive stem cell therapy; applying shockwave therapy to the penis; developing a platelet-rich plasma preparation from the patient; introducing a stem cell specimen into a vial having a needle-free injector; introducing the platelet-rich plasma preparation into the vial; injecting, through the needle-free injector, the stem cell specimen and platelet-rich plasma preparation directly on penile surface from multiple positions, multiple depths, and multiple angles; reapplying shockwave therapy to the penis after a duration; and monitoring the patient for a post-procedure duration after administration of the stem cell specimen to the penis.

### FIELD OF THE INVENTION

The present invention relates generally to a Zeus protocol method for enhancing male sexual wellness with shockwave therapy and stem cells. More so, the present invention relates to a method for enhancing male sexual wellness with shockwave therapy and stem cells; whereby the method promotes tissue repair and regeneration in or near the genitalia, i.e., male penis, through the combinative effects of shockwave therapy and needle-free injection of a stem cell specimen directly on the penis; and whereby needle-free administration of stem cells allows for a low anxiety, painless entry, and a more uniform distribution of the deposited stem cell specimen.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

It is known that the genitalia, or sex organ, is a part of the body that is involved in sexual reproduction. The reproductive organs together constitute the reproductive system. The testis in the male is one of the primary sex organs. The penis is the primary sexual organ that male animals use to inseminate sexually receptive mates (usually females and hermaphrodites) during copulation. The penis is made of several parts: the glans (head) of the penis; the corpus cavernosum, which includes two columns of tissue running along the sides of the penis; the corpus spongiosum, which includes a column of sponge-like tissue running along the front of the penis and ending at the glans penis, and which fills with blood during an erection, keeping the urethra which runs through the corpus spongiosum, conducting urine out of the body.

Typically, stem cells are primitive cells in our body serving as a reservoir, able to replenish itself and differentiate into a wide range of specialized cells, in order to replace damaged cells and regenerate tissue. Stem cells have innate intelligence, able to home in to injured areas, secrete bioactive molecules that exert local and systemic effects, reduce inflammation, and recruit local cells to assist in the healing process. One common stem cell specimen is a mesenchymal stem cell.

Other proposals have involved methods for treating the penis with stem cell specimens. The problem with these methods is that they do not incorporate shockwave therapy with the stem cells. Also, the prior art methods do not inject mesenchymal stem cells directly on the penile surface. Even though the above cited methods for administering stem cell specimens meet some of the needs of the market, a method for enhancing male sexual wellness with shockwave therapy and stem cells to promote tissue repair and regeneration in or near the male penis through the combinative effects of shockwave therapy and needle-free injection of a stem cell specimen directly on the genital, is still desired.

### SUMMARY

Illustrative embodiments of the disclosure are generally directed to a Zeus protocol method for enhancing male sexual wellness with shockwave therapy and stem cells. The method promotes tissue repair and regeneration in or near the genitalia, i.e., male penis, through the combinative effects of shockwave therapy and needle-free injection of a stem cell specimen directly on the penile surface. The needle-free administration of stem cells allows for a low anxiety, painless entry, and a more uniform distribution of the deposited stem cell specimen. The method may be helpful in conditions such as inorgasmia, dyspareunia, urinary incontinence, erectile dysfunction, and may improve male sexual arousal and quality of orgasms.

In one embodiment, the method involves a series of steps, including: preparing the patient, both mentally and physically to receive stem cell therapy; applying shockwave therapy to the penis; developing a platelet-rich plasma preparation from the patient; introducing a stem cell specimen into a vial having a needle-free injector; introducing the platelet-rich plasma preparation into the vial.

The method also includes: injecting, through the needle-free injector, the stem cell specimen and the platelet-rich plasma preparation directly on or near the penis from multiple positions, multiple depths, and multiple angles; reapplying shockwave therapy to the penis after a duration; and monitoring the patient for a post-procedure duration after administration of the stem cell specimen and the platelet-rich plasma preparation.

The method for enhancing male sexual wellness with shockwave therapy and stem cell therapy includes an initial Step of obtaining consent from the patient to proceed with the method.

The method may include an initial Step of obtaining at least one metric of a patient.

The method may further comprise a Step of applying shockwave therapy to the penis of the patient.

A Step includes developing a platelet-rich plasma preparation from the patient.

In some embodiments, a Step comprises obtaining a stem cell specimen.

A Step includes introducing the stem cell specimen into a vial, the vial having a detachably attached needle-free injector defined by multiple nozzles.

In some embodiments, a Step may include introducing the platelet-rich plasma preparation into the vial.

A Step comprises drawing about 0.5 cubic centimeters of the stem cell specimen and the platelet-rich plasma preparation from the vial into each nozzle of the needle-free injector.

The method may further comprise a Step of injecting, through at least one of the nozzles of the needle-free injector, the stem cell specimen and the platelet-rich plasma preparation directly on or near the penis from multiple positions, multiple depths, and multiple angles, whereby multiple injections are performed.

A Step includes reapplying, after a duration, the shockwave therapy to the penis.

A final Step includes monitoring the patient for a post-procedure duration after administration of the stem cell specimen and the platelet-rich plasma preparation.

In another aspect, the method further comprises a step of activating the platelet-rich plasma by positioning the platelet-rich plasma in a 37° Fahrenheit water bath for 1 to 3 minutes.

In another aspect, the method further comprises a step of applying an ultrasound gel over the penis.

In another aspect, the method further comprises a step of applying a numbing cream over the penis.

In another aspect, the method further comprises a step of heating a stem cell specimen vial containing the stem cell specimen to induce thawing by emerging the stem cell specimen vial in a 37° water bath, or by rolling the stem cell specimen vial against the palms of the hands.

In another aspect, the step of introducing the stem cell specimen into a vial, further comprises drawing 2 cubic centimeters of the stem cell specimen into a 5 cubic centimeter syringe, the syringe being detachably attached to a 18 G 1½″ needle.

In another aspect, the step of introducing the platelet-rich plasma preparation into the vial, further comprises drawing 1 cubic centimeter of the platelet-rich plasma preparation into the 5 cubic centimeter syringe, whereby the stem cell specimen and the platelet-rich plasma preparation are mixed.

In another aspect, the method further comprises a step of puncturing a rubber top on the vial with the needle-free injector.

In another aspect, the needle-free injector comprises six nozzles.

In another aspect, the genitalium comprises a penis.

In another aspect, the method further comprises a step of cleaning the penis with a povodone-iodine swabstick.

In another aspect, the step of injecting, through the needle-free injector, the stem cell specimen and the platelet-rich plasma preparation directly on or near the genitalium from multiple positions, multiple depths, and multiple angles, further comprises injecting, through at least one of the nozzles of the needle-free injector, the stem cell specimen and the platelet-rich plasma preparation at a two o'clock position at the proximal base of the penile shaft, at the mid-length of the penile shaft, and near the distally disposed glans of the penis.

In another aspect, the step of injecting, through the needle-free injector, the stem cell specimen and the platelet-rich plasma preparation directly on or near the genitalium from multiple positions, multiple depths, and multiple angles, further comprises injecting, through at least one of the nozzles of the needle-free injector, the stem cell specimen and the platelet-rich plasma preparation at a ten o'clock position at the proximal base of the penile shaft, at the mid-length of the penile shaft, and near the distally disposed glans of the penis.

One objective of the present invention is to use stem cells to help repair damaged tissues and repair injuries near the penis.

Another objective is to use a shockwave device to generate shock waves which can be adjusted for intensity, frequency and modulation so as to maximize therapeutic effects.

Another objective is to reduce local inflammation, improve blood flow, promote new blood vessel formation, facilitate breaking down of scar tissue, stimulate growth factor release, and activate local stem cell population.

Another objective is to promoting tissue regeneration in and around the penis.

Another objective is to prevent infections in the penis through use of a Povidone-iodine swab sticks.

Another objective is to ensure the mental well-being of the patient prior to the stem cell therapy.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a flowchart of an exemplary Zeus protocol method for enhancing male sexual wellness with shockwave therapy and stem cells, in accordance with an embodiment of the present invention;

FIG. 2 illustrates a sectioned side view of the male penis fully extended, in accordance with an embodiment of the present invention;

FIG. 3 illustrates a sectioned view of the male penis at an angle, in accordance with an embodiment of the present invention;

FIG. 4 illustrates a perspective view of the male penis fully extended, showing the injection points at the proximal base of the penile shaft, the mid-length of the penile shaft, and the shaft distal end, for both the 10 o'clock and 2 o'clock positions, in accordance with an embodiment of the present invention; and

FIG. 5 illustrates a perspective view of the male penis being injected with stem cell specimen with a needle-free injector, in accordance with an embodiment of the present invention.

Like reference numerals refer to like parts throughout the various views of the drawings.

### DETAILED DESCRIPTION OF THE INVENTION

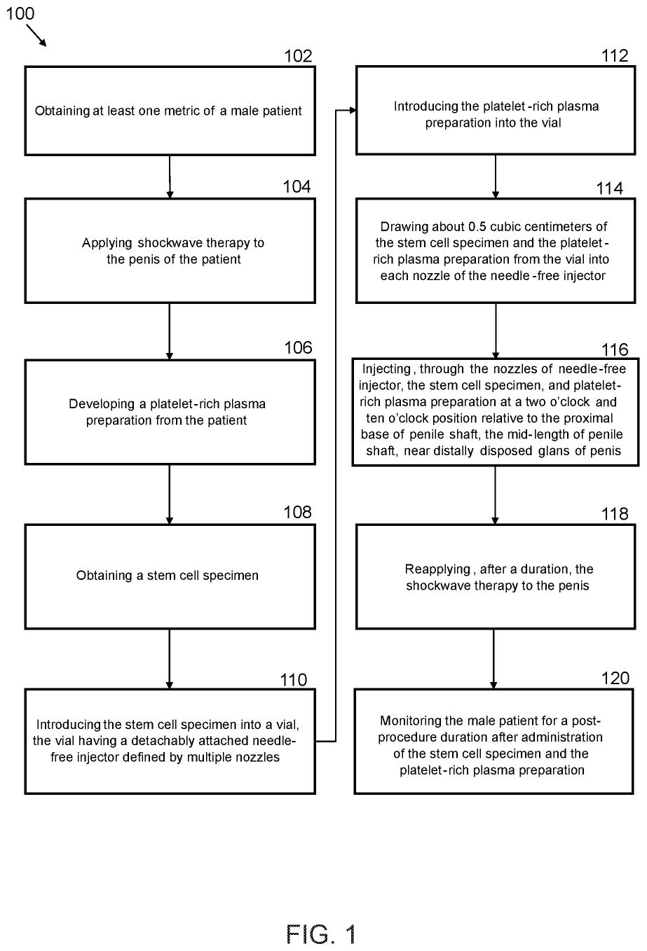

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

A Zeus protocol method 100 for enhancing male sexual wellness with shockwave therapy and stem cells is referenced in FIGS. 1-5. The method 100 promotes tissue repair and regeneration in or near the genitalia, such as the penis. The method 100 is operable with the male penis. The method 100 creates a synergy between shockwave therapy and needle-free injection of a stem cell specimen directly on the penis. It is unique that the needle-free administration of stem cells allows for a low anxiety, painless entry, and a more uniform distribution of the deposited stem cell specimen. The method 100 is efficacious for healing conditions, such as inorgasmia, dyspareunia, urinary incontinence, erectile dysfunction, and may improve sexual arousal and quality of orgasms in the penis.

The method 100 generally includes a series of steps that both prepare the genitalia/penis for shockwave therapy and stem cell deposition. The method 100 includes: preparing the patient, both mentally and physically to receive stem cell therapy; applying shockwave therapy to the genitalium; developing a platelet-rich plasma preparation from the patient; introducing a stem cell specimen into a vial having a needle-free injector; and introducing the platelet-rich plasma preparation into the vial.

The method 100 also includes injecting, through the needle-free injector, the stem cell specimen and the platelet-rich plasma preparation directly on or near the genitalium from multiple positions, multiple depths, and multiple angles; reapplying shockwave therapy to the penis after a duration; and monitoring the patient for a post-procedure duration after administration of the stem cell specimen and the platelet-rich plasma preparation.

The method 100 is effective for reducing local inflammation, improving blood flow, promoting new blood vessel formation, facilitating breaking down of scar tissue, stimulating growth factor release, and activating local stem cell population. The method 100 is also efficacious for promoting tissue regeneration in and around the penis. Other benefits include using stem cells to help repair damaged tissues and repair injuries near the penis; and using a shockwave device to generate shock waves which can be adjusted for intensity, frequency and modulation so as to maximize therapeutic effects.

The method 100, as described below, requires a unique needle-free injector with multiple nozzles, stem cell specimens, topical creams, and syringes to properly administer the stem cell specimen and the platelet-rich plasma preparation on the penis. One requirement can include a ChareCore™ stem cell specimen. In one embodiment, the ChareCore™ can be 2 cc of the actual stem cell specimen having a substantial amount of mesenchymal stem cells.

It is known in the art that CharaCore™ contains 8-10 million cells/cc. These are “native stem cells.” Research shows that native mesenchymal stem cells are at least 10 times more potent than expanded MSC's. Further, Characore™ exhibits a high cell count, with valuable components from umbilical cord blood, cord tissue and amniotic membrane. Delicately processed, thus maximally preserving therapeutic elements. Characore™ contains a high amount of MSC's and various growth factors. Characore™ is offered in 1 cc and 2 cc vials for all forms of injections; with the present invention utilizing the 2 cc vial.

The method 100 also requires an ultrasound gel for the shockwave device, and a BLT (Benzocaine, Lidocaine, Tetracaine) topical anesthetic cream for numbing the treatment area around the penis. For administrating the stem cell specimen and the platelet-rich plasma preparation, the method 100 utilizes one Lenis needle-free injector (or 2 for fasting loading); one Lenis adapter for drawing liquid into six nozzles; and six Lenis injector nozzles.

The method 100 also contains the stem cell specimen and the platelet-rich plasma preparation in a 10 cc sterile empty vial with rubber stopper, and injects through a 5 cc syringe and an 18 G 1½″ needle. One objective of the method 100 is to prevent infections on the penis. Thus, the method 100 may also require a Povidone-iodine swabsticks and alcohol swabs to clean the penis and penile surface.

The present method 100 can generate revenue in numerous ways. For example, in order for a healthcare provider to provide treatment for the male sexual wellness treatment (Zeus Lift), or the intranasal procedure (Brain Reboot), they will have to pay a licensing fee of: $100 per month as long as they are providing treatment or using those trademarked phrases on their website or other marketing material.

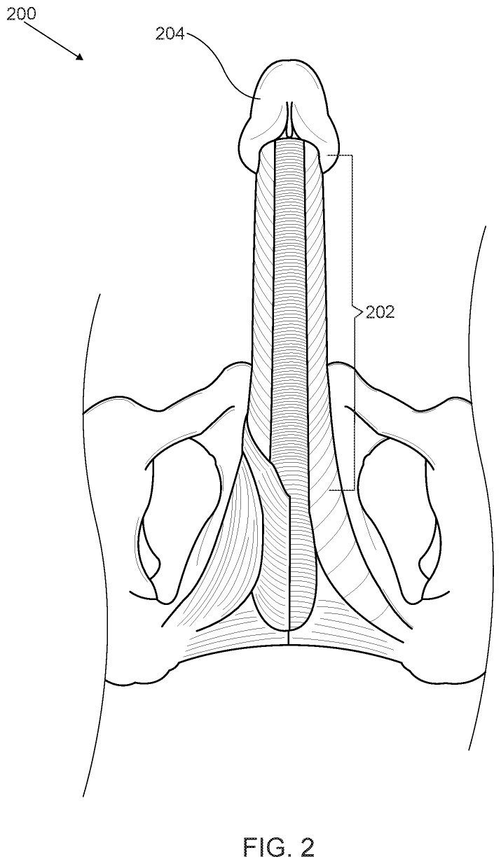

FIG. 1 illustrates a flowchart diagram of an exemplary method 100 for enhancing male sexual wellness with shockwave therapy and stem cells. The method 100 is operable with a male penis 200 (FIG. 2) to enhance wellness thereto. The method 100 may include an initial Step 102 of obtaining at least one metric of a male patient. The metric of a patient may include, without limitation, body temperature, blood pressure, and pulse oximetry measurement. Though in other embodiments, different metrics may be measured.

In some embodiments, the method 100 also includes a step of obtaining consent to proceed with the method 100 from the patient. Standard consent forms commonly used in the medical industry may be used for this purpose. In one embodiment, the method 100 requires a clinical evaluation of the patient to determine appropriateness for shockwave and stem cell therapy to the penis. The clinic collects a detailed medical history and provides a physical exam of the patient.

In order to prepare the penis for application of a shockwave device, the method comprises a step of applying an ultrasound gel over the penile surface 306. In one embodiment, the ultrasound gel is applied over the entire penis area. The shock wave device is positioned over the penis. A medium intensity is applied with intermittent high intensity as tolerated by the patient, for 15 minutes using a slow circular motion.

Further, a numbing cream is applied over the penis to reduce pain and swelling during stem cell deposition. This requires cleaning off the ultrasound gel, and applying a BLT numbing cream on the penile surface 306 along the entirety of the penis shaft. The topical anesthetic numbing cream is allowed to remain for about 30 minutes, so as to be absorbed by the penis sufficiently.

The method 100 may further comprise a Step 104 of applying shockwave therapy to the penis of the patient. This can be performed with a shockwave device. Those skilled in the art will recognize that shockwave treatment works by using radial acoustic shockwaves to stimulate self-healing. The generated shockwave goes through the tissue and stimulates the cells dealing with connective tissue and bone healing. By stimulating these cells, the body's natural self-healing mechanism is triggered.

In one embodiment, the method has an Initial Phase that requires two sessions for maximal results, about 8-12 weeks apart. In each session, 2 cc CharaCore™ is required. Ideally, shockwave therapy is recommend immediately prior to stem cell injections, for ten minutes at medium strength. This can be applied twenty-four hours after stem cell injections, and then two times per week for six weeks, apply shockwave therapy using in-home device. Each time, a medium intensity shockwave is applied. However, the shockwave can also be alternated with high intensity shockwaves as the patient can tolerate, for about 15 minutes.

A Step 106 includes developing a platelet-rich plasma preparation from the patient. This may involve drawing blood from the patient through intravenous means to prepare for platelet-rich plasma. This process could take approximately 20 minutes. In one non-limiting embodiment, the platelet-rich plasma is activated by placing in a 37° water bath for 1-3 minutes. In one alternative embodiment, the method includes a step of activating the platelet-rich plasma by positioning the platelet-rich plasma in a 37° Fahrenheit water bath for 1 to 3 minutes.

In some embodiments, a Step 108 comprises obtaining a stem cell specimen. In one embodiment, the stem cell specimen is in a stem cell specimen vial, which is stored in dry ice. In another exemplary collection of the stem cell specimen, the stem cell specimen is removed from dry ice, and a peel off product sticker is affixed to a medical record file. The stem cell specimen is prepared for use by heating a stem cell specimen vial containing the stem cell specimen. This induces thawing by emerging the stem cell specimen vial in a 37° Fahrenheit water bath, or by rolling the stem cell specimen vial against the palms of the hands. Either manner heats the stem cell specimen sufficiently for pouring into a vial, as described below. The stem cell specimen is the content by putting vial in 37° Celsius water bath (or by holding vial in hand, gently rolling against skin). It generally takes 2-2½ minutes for complete thawing via water bath, and 4-5 minutes by hand.

A Step 110 includes introducing the stem cell specimen into a vial, the vial having a detachably attached needle-free injector defined by multiple nozzles 500. In some embodiments, 2 cubic centimeters of the stem cell specimen is drawn into a 5 cubic centimeter syringe, the syringe being detachably attached to an 18 G 1½″ needle. Also, 1 cubic centimeter of the platelet-rich plasma preparation is drawn into the 5 cubic centimeter syringe. In this manner, the stem cell specimen and the platelet-rich plasma preparation are mixed in the vial.

Further, the needle-free injector comprises one Lenis adapter for drawing liquid into six nozzles; and six Lenis injector nozzles. Though other types of nozzles may be used. As described below, each nozzle will administer a dosage of stem cell specimen and platelet-rich plasma preparation to a different section, angle, and depth of the penis.

In some embodiments, a Step 112 may include introducing the platelet-rich plasma preparation into the vial. It is significant to note that the vial may have a rubber cap; and thereby require a step of puncturing a rubber top on the vial with the needle-free injector. Thus, it is necessary to puncture the rubber top of the vial with the needle-free adapter, and make sure adapter is sitting securely over the vial.

A Step 114 comprises drawing about 0.5 cubic centimeters of the stem cell specimen and the platelet-rich plasma preparation from the vial into each nozzle of the needle-free injector. The needle-free injector is cocked to the side and a pre-filled nozzle is attached into tip of the injector. It is necessary to attach the nozzle securely onto the adapter, and invert the vial. Then, 0.5 cc of stem cell prep is drawn into each of the 6 nozzles. In some embodiments the method includes cleaning the vulval area of the penile surface with a povodone-iodine swabstick 302, along the entirety of the shaft.

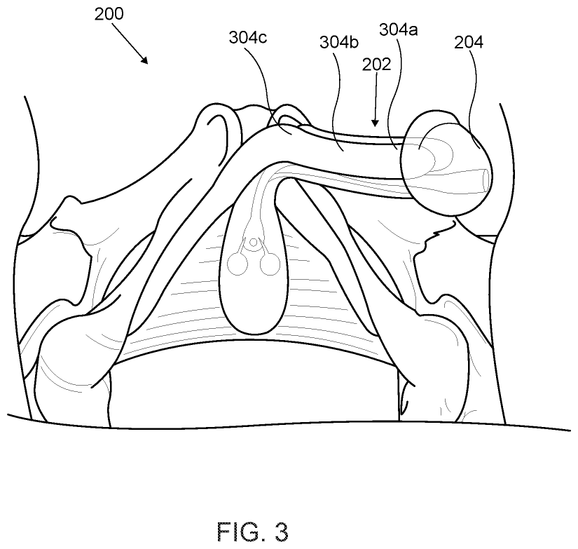

Another Step for male sexual wellness includes extending the penile shaft in preparation for stem cell deposition along the penile surface 306 (See FIG. 3). The fingers of the medical professional can grip the distal gland and pull the penis shaft outwardly.

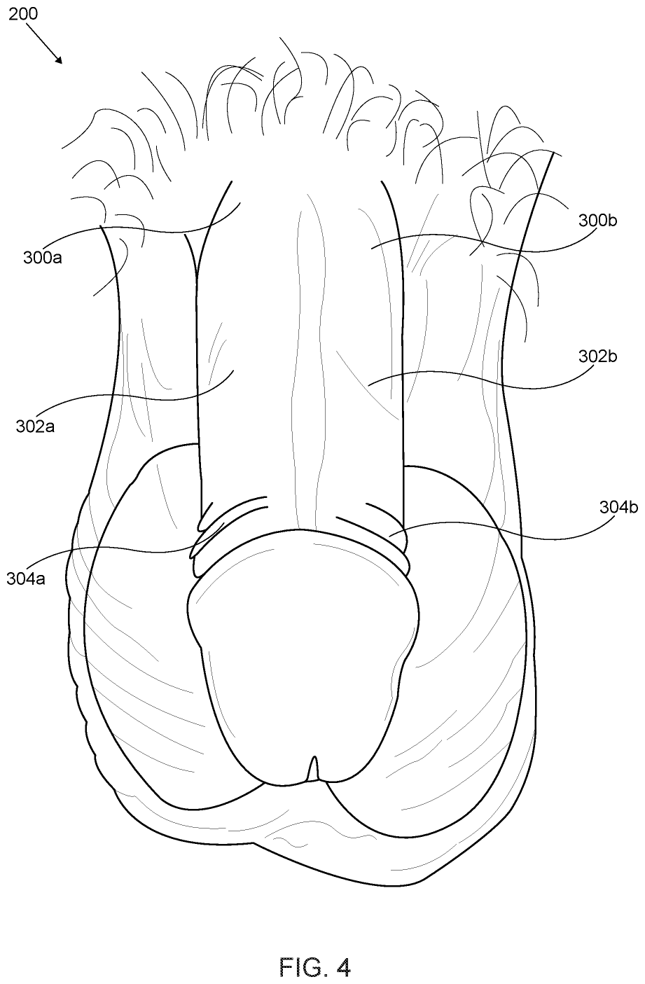

The method 100 may further comprise injecting, through at least one of the nozzles of the needle-free injector, the stem cell specimen and the platelet-rich plasma preparation directly on or near the penis from multiple positions, multiple depths, and multiple angles, whereby multiple injections are performed. Looking again at FIG. 4, and correlating Step 116, the method 100 more specifically involves injecting, through at least one of the nozzles of the needle-free injector, the stem cell specimen and the platelet-rich plasma preparation at a two o'clock position and a ten o'clock position relative to the proximal base of the penile shaft, the mid-length of the penile shaft, and near the distally disposed glans 204 of the penis 200.

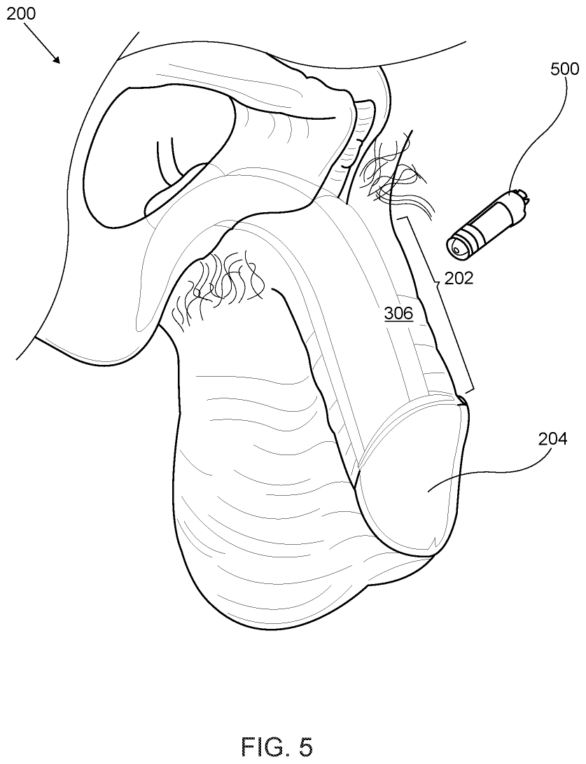

After this initial injection, subsequent injections include injecting, through at least one of the nozzles, the stem cell specimen and the platelet-rich plasma preparation are oriented at a ten o'clock position and two o'clock positions relative to the proximal base 300a, 300b of the penile shaft 202, the mid-length 302a, 302b of the penile shaft, and the shaft distal end 304a, 304b, near the glans of the penis relative to the proximal base 300a of the penile shaft 202, the mid-length 302a of the penile shaft, and the shaft distal end 304a, near the glans 204 of the penis 200 (FIG. 5).

This series of injections are performed twice, for both lateral sides of the penis shaft, which is the ten o'clock position and the two o'clock position. Thus, the left side proximal base 300a and right side proximal base 300b; the left side mid-length 302a and the right side mid-length 302b, and the shaft distal end 304a and right side shaft distal end 304b are injected with separate nozzles 500 carrying 0.5 cubic centimeters of the stem cell specimen and the platelet-rich plasma preparation. In this manner, injections occur at the two o'clock position relative to the proximal base 300b of the penile shaft 202, the mid-length 302b of the penile shaft, and near the distally disposed glans 304c of the penis 200.

After complete deposition of stem cell specimen on the penile surface, the patient uses the shockwave device at home twenty-four hours after treatment, and three times per week thereafter for six weeks. A Step 118 includes reapplying, after a duration, the shockwave therapy to the penis. In one embodiment, the patient uses the shockwave device on the penis while at home, about twenty-four hours after treatment, and three times per week thereafter for six weeks.

For example, a shockwave home session instructions include: applying ultrasound gel over entire penile sureface; placing the head of shockwave device over entire areas for a total of 15 minutes; applying medium intensity with intermittent high intensity as tolerated; all while using a very slow circular motion with the shockwave device. As in Step 104, the generated shockwave passes through the tissue and stimulates the cells dealing with connective tissue and bone healing.

A final Step 120 includes monitoring the male patient for a post-procedure duration after administration of the stem cell specimen and the platelet-rich plasma preparation. The date of a maintenance session after administration of the stem cell specimen and platelet-rich plasma preparation depends on how long the sexual enhancement is sustained. This depends on the patient's age, health condition and lifestyle factors. However, intervals could range between about 1 to 3 years. Each maintenance session follows the same procedure as one of above sessions.

Although the process-flow diagrams show a specific order of executing the process steps, the order of executing the steps may be changed relative to the order shown in certain embodiments. Also, two or more blocks shown in succession may be executed concurrently or with partial concurrence in some embodiments. Certain steps may also be omitted from the process-flow diagrams for the sake of brevity. In some embodiments, some or all the process steps shown in the process-flow diagrams can be combined into a single process.

It is recognized in the art that stem cell therapy is a complex and potentially dangerous procedure. Thus, the method 100 may further include alternative Steps of providing emergency supplies in the event of untoward reactions. It may also be beneficial to provide Epinephrine 1:1000 (1 mg/ml), inject 0.5 mg IV or IM in case of anaphylactic reaction. The Epinephrine administration may repeat in 5 minutes if needed.

Yet another emergency supply for the penis is Benadryl 50 mg IV, which can be pushed over 2 minutes for anaphylactic reactions (may give after epinephrine dose). Finally, an Albuterol inhaler is provided in case the patient demonstrates shortness of breath during or after the method is applied. However, the method 100 may also utilize other emergency systems and processes known in the art for adverse penis reactions to the treatment.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A method for enhancing male sexual wellness with shockwave therapy and stem cells, the method comprising:
obtaining at least one metric of a male patient;
applying shockwave therapy to the penis of the male patient;
developing a platelet-rich plasma preparation from the male patient;
obtaining a stem cell specimen;
introducing the stem cell specimen into a vial, the vial having a detachably attached needle-free injector defined by multiple nozzles;
introducing the platelet-rich plasma preparation into the vial;
drawing about 0.5 cubic centimeters of the stem cell specimen and the platelet-rich plasma preparation from the vial into each nozzle of the needle-free injector;
injecting, through at least one of the nozzles of the needle-free injector, the stem cell specimen and the platelet-rich plasma preparation directly on or near the penis from multiple positions, multiple depths, and multiple angles, whereby multiple injections are performed;
reapplying, after a duration, the shockwave therapy to the penis; and
monitoring the patient for a post-procedure duration after administration of the stem cell specimen and the platelet-rich plasma preparation.

2. The method of claim 1, further comprising a step of obtaining consent from the male patient to proceed with the method.

3. The method of claim 1, further comprising a step of activating the platelet-rich plasma by positioning the platelet-rich plasma in a 37 degree Fahrenheit water bath for 1 to 3 minutes.

4. The method of claim 1, further comprising a step of applying an ultrasound gel over the penis.

5. The method of claim 4, further comprising a step of applying a numbing cream over the penis.

6. The method of claim 1, further comprising a step of heating a stem cell specimen vial containing the stem cell specimen to induce thawing by emerging the stem cell specimen vial in a 37° water bath, or by rolling the stem cell specimen vial against the palms of the hands.

7. The method of claim 1, wherein the step of introducing the stem cell specimen into a vial, further comprises drawing 2 cubic centimeters of the stem cell specimen into a 5 cubic centimeter syringe, the syringe being detachably attached to a 18 G 1½″ needle.

8. The method of claim 7, wherein the step of introducing the platelet-rich plasma preparation into the vial, further comprises drawing 1 cubic centimeter of the platelet-rich plasma preparation into the 5 cubic centimeter syringe, whereby the stem cell specimen and the platelet-rich plasma preparation are mixed.

9. The method of claim 8, further comprising a step of puncturing a rubber top on the vial with the needle-free injector.

10. The method of claim 9, wherein the needle-free injector comprises six nozzles.

11. The method of claim 10, wherein the step of injecting, through the needle-free injector, the stem cell specimen and the platelet-rich plasma preparation directly on or near the penis from multiple positions, multiple depths, and multiple angles, further comprises: injecting, through at least one of the nozzles of the needle-free injector, the stem cell specimen and the platelet-rich plasma preparation at a two o'clock position relative to the proximal base of the penile shaft, the mid-length of the penile shaft, and near the distally disposed glans of the penis.

12. The method of claim 11, wherein the step of injecting, through the needle-free injector, the stem cell specimen and the platelet-rich plasma preparation directly on or near the penis from multiple positions, multiple depths, and multiple angles, further comprises: injecting, through at least one of the nozzles of the needle-free injector, the stem cell specimen and the platelet-rich plasma preparation are oriented at a ten o'clock position relative to the proximal base of the penile shaft, the mid-length of the penile shaft, and the shaft distal end, near the glans of the penis.

13. The method of claim 12, further comprising injecting, through at least one of the nozzles of the needle-free injector, the stem cell specimen and the platelet-rich plasma preparation are oriented at a two o'clock position relative to the proximal base of the penile shaft, the mid-length of the penile shaft, and the shaft distal end, near the glans of the penis.

14. A method for enhancing male sexual wellness with shockwave therapy and stem cells, the method comprising:
obtaining at least one metric of a male patient;
applying shockwave therapy to the penis of the male patient;
developing a platelet-rich plasma preparation from the male patient;
obtaining a stem cell specimen;
introducing the stem cell specimen into a vial, the vial having a detachably attached needle-free injector defined by multiple nozzles;
introducing the platelet-rich plasma preparation into the vial;
drawing about 0.5 cubic centimeters of the stem cell specimen and the platelet-rich plasma preparation from the vial into each nozzle of the needle-free injector;
injecting, through at least one of the nozzles of the needle-free injector, the stem cell specimen and the platelet-rich plasma preparation at a two o'clock position relative to the proximal base of the penile shaft, the mid-length of the penile shaft, and near the distally disposed glans of the penis;
injecting, through at least one of the nozzles of the needle-free injector, the stem cell specimen and the platelet-rich plasma preparation are oriented at a ten o'clock position relative to the proximal base of the penile shaft, the mid-length of the penile shaft, and the shaft distal end, near the glans of the penis;
reapplying, after a duration, the shockwave therapy to the penis; and
monitoring the patient for a post-procedure duration after administration of the stem cell specimen and the platelet-rich plasma preparation.

15. The method of claim 14, further comprising a step of activating the platelet-rich plasma by positioning the platelet-rich plasma in a 37 degree Fahrenheit water bath for 1 to 3 minutes.

16. The method of claim 15, further comprising a step of applying an ultrasound gel over the penis.

17. The method of claim 16, further comprising a step of applying a numbing cream over the penis.

18. The method of claim 17, further comprising a step of heating a stem cell specimen vial containing the stem cell specimen to induce thawing by emerging the stem cell specimen vial in a 37° water bath, or by rolling the stem cell specimen vial against the palms of the hands.

19. The method of claim 18, wherein the step of introducing the stem cell specimen into a vial, further comprises drawing 2 cubic centimeters of the stem cell specimen into a 5 cubic centimeter syringe, the syringe being detachably attached to a 18 G 1½″ needle.

20. A method for enhancing male sexual wellness with shockwave therapy and stem cells, the method comprising:
obtaining at least one metric of a male patient;
applying shockwave therapy to the penis of the male patient;
developing a platelet-rich plasma preparation from the male patient;
obtaining a stem cell specimen;
introducing about 2 cubic centimeters of the stem cell specimen into a vial having a detachably attached needle-free injector, the needle-free infector comprising six nozzles;
introducing about 1 cubic centimeter the platelet-rich plasma preparation into the vial;
drawing about 0.5 cubic centimeters of the stem cell specimen and the platelet-rich plasma preparation into each nozzle of the needle-free injector;
cleaning the penile surface of the penis with a povodone-iodine swabstick;
fixedly extending the penis shaft of the penis;
injecting, through at least one of the nozzles of the needle-free injector, the stem cell specimen and the platelet-rich plasma preparation at a two o'clock position relative to the proximal base of the penile shaft, the mid-length of the penile shaft, and near the distally disposed glans of the penis;
injecting, through at least one of the nozzles of the needle-free injector, the stem cell specimen and the platelet-rich plasma preparation are oriented at a ten o'clock position relative to the proximal base of the penile shaft, the mid-length of the penile shaft, and the shaft distal end, near the glans of the penis;
injecting, through at least one of the nozzles of the needle-free injector, the stem cell specimen and the platelet-rich plasma preparation are oriented at a two o'clock position relative to the proximal base of the penile shaft, the mid-length of the penile shaft, and the shaft distal end, near the glans of the penis;
reapplying, after a duration, the shockwave therapy to the penis; and
monitoring the patient for a post-procedure duration after administration of the stem cell specimen and the platelet-rich plasma preparation.
